(19) United States
(12) Patent Application Publication
(10) Pub. No.: US 2009/0170094 A1
(43) Pub. Date: Jul. 2, 2009

(54) FLUORESCENT LABELING SUBSTANCE COMPRISING NANOPARTICLES OR NANORODS

(76) Inventors: Mitsuru Sekiguchi, Tokyo (JP); Kazuyoshi Goan, Tokyo (JP); Kazuya Tsukada, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/162,157

(22) PCT Filed: Nov. 27, 2006
(86) PCT No.: PCT/JP2006/323560

§ 371 Date: Jul. 25, 2008

(30) Foreign Application Priority Data
Jan. 30, 2006 (JP) 2006-021391

(51) Int. Cl. C12Q 1/68

(52) U.S. Cl. 435/6; 977/773

(57) ABSTRACT

A fluorescent labeling substance that is capable of realizing highly appropriate labeling through enhancing of the luminous efficiency of semiconductor nanoparticles or nanorods. The fluorescent labeling substance can be provided by disposing on a surface of shell of nanorods or nanoparticles having a modification group capable of adsorption with a biosubstance, such as protein, nucleic acid or antibody, a region devoid of the above modification group.

### TECHNICAL FIELD

The present invention relates to a fluorescent labeling substance which comprises core/shell nanoparticles or core/shell nanorods and is used for analysis targeting biosubstances.

### TECHNICAL BACKGROUND

Nanoparticles are applied to the bio-field as a fluorescent labeling substance used for analysis of the behavior of different genes proteins in a cell which have been dyed with plural colors (as described in non-patent document 1). FIGS. 1(a) and 1(b) illustrate examples of a semiconductor nanoparticle used in such a field and having a core/shell structure. On the overall surface of this semiconductor nanoparticle is formed a surface modification layer 3 comprised of a molecule containing a carboxyl group to introduce, to the semiconductor nanoparticles, a lectin as an antigen which is capable of specifically recognizing a sugar chain existing on the cancer cell surface.

When using a semiconductor nanoparticle exhibiting such behavior, the lectin is generally introduced onto the overall particle surface (FIG. 2). In fact, a lectin which has been introduced to a portion not bonding to a cancer cell is unnecessary one which does not perform its function (FIG. 3). Covering the surface with the surface modification layer and lectin produces problems such that an incident exciting light (ultraviolet ray) or a fluorescence emitted from the semiconductor nanoparticle is absorbed by the surface modification layer and lectin, leading to substantially reduced light-emitting efficiency (FIGS. 4 and 5).

There was proposed a semiconductor nanoparticle surface-modified with a compound containing a hydrophilic functional group, for use as a fluorescent labeling substance having enhanced hydrophilicity and causing no coagulation in an aqueous solution. However, such a semiconductor nanoparticle is modified on its entire surface and problems arise with the light-emitting efficiency, as described above.
Patent document 1: U.S. Pat. No. 6,251,303
Non-patent document 1: Baba, Oyo Butsuri vol. 74 (2005), pp. 1543-1554

### DISCLOSURE OF THE INVENTION

### Problem to be Solved

It is an object of the invention to provide a labeling substance which is capable of realizing more suitable labeling through enhancement of light-emitting efficiency of a semiconductor nanoparticle or nanorod.

### Means for Solving the Problem

The present invention has come into being, based on the discoveries by the inventors of this application that in at least a part of the shell surface of a semiconductor nanoparticle or nanorod having a modification group capable of being adsorbed to a biosubstance, for example, an antigen such as a sugar chain existing on the cancer cell surface, a protein or a nucleic acid, a region of such a modification group being not present was provided, and thereby was obtained a fluorescent labeling substance exhibiting enhanced efficiencies of exciting light entering to and fluorescence emitting from semiconductor nanoparticles or nanorods.

The area of the above-described region having no modification group preferably accounts for not less than 50% of the total shell surface. For example, a modification group capable of adsorbing a biosubstance is allowed to exist on only one half of the spherical surface of a nanoparticle, only one half of the cylindrical surface of a nanorod, or only the upper surface or only the bottom surface of a nanorod, rendering it feasible to secure an area of such a region.

The modification group capable of being adsorbed to a biosubstance is introduced as follows; a COOH (carboxyl) group or a NH₂ (amino) group is formed on the surface of a nanoparticle or nanorod by carboxylation (COOH formation) of an alkyl group such as a CH₃ group or C having an unbonded bond, contained in SiC, SiOCH, SiCNH or the like or by forming a substance containing a NH₂ group, and then, the thus formed carboxy group or amino group is allowed to react with a modification group.

Preferably, the nanoparticle or nanorod of the invention is a core/shell one comprising a core composed of a semiconductor nanocrystal and a shell composed of a substance having a greater band gap than the core, and the average particle diameter of nanoparticles or the average diameter of nanorods is desirably from 2 to 50 nm.

### EFFECT OF THE INVENTION

According to the invention, there is provided a fluorescent labeling substance with enhanced substantial light-emitting efficiency and capable of performing highly precise analysis in the field of targeting biosubstances, such as detection of cancer cells.

### BRIEF DESCRIPTION OF THE DRAWINGS

FIG. 1 illustrates prior art: semiconductor nanoparticle of a core/shell structure,

FIG. 2 illustrates prior art: semiconductor nanoparticle having lectin introduced,

FIG. 3 illustrates prior art: semiconductor nanoparticle bonded to a cancer cell,

FIG. 4 illustrates prior art: incidence of an exciting light onto semiconductor nanoparticle bonded to a cancer cell,

FIG. 5 illustrates prior art: fluorescence emission from semiconductor nanoparticle bonded to a cancer cell,

FIG. 6 illustrates the invention (Example 1) using SiOCH: preparation of nanoparticle and introduction of a carboxy group,

FIG. 7 illustrates the invention (Example 1) using SiOCH: introduction of lectin and bonding to a cancer cell,

FIG. 8 illustrates the invention (Example 1) using SiOCH: exposure of a fluorescent labeling substance bonded to a cancer cell to exciting light and its fluorescence emission,

FIG. 9 illustrates the invention (Example 2) using SiNH: preparation of nanoparticle and introduction of an amino group,

FIG. 10 illustrates the invention (Example 2) using SiNH: introduction of lectin and bonding to a cancer cell,

FIG. 11 illustrates the invention (Example 2) using SiNH: exposure of a fluorescent labeling substance bonded to a cancer cell to exciting light and its fluorescence emission,

FIG. 12 illustrates the invention (Example 4) using Si/SiO₂: preparation of nanorod,

FIG. 13 illustrates the invention (Example 4) using Si/SiO₂: introduction of surface-modification group,

FIG. 14 illustrates the invention (Example 5) using silanol (silane coupling agent).

### DESCRIPTION OF DESIGNATION

1: Core (CdSe)
2: Shell (ZnS)
3: Surface modification layer
4: Lectin
5: Cancer cell
6: Normal cell
7: Observation instrument and observer
11: Si
12: Sio₂
13: SiOCH layer
33: SiNH layer
40: Polystyrene ball
41: Si substrate
42: Si rod
43: SiO₂
44: Si
45: Si/SiO₂ nanorod
46: SiNH
51: COOH—Si(OCH₃)₃ solution

### PREFERRED EMBODIMENT OF THE INVENTION

### Nanoparticle and Nanorod

### TECHNICAL FIELD

The present invention relates to a fluorescent labeling substance which comprises core/shell nanoparticles or core/shell nanorods and is used for analysis targeting biosubstances.

### TECHNICAL BACKGROUND

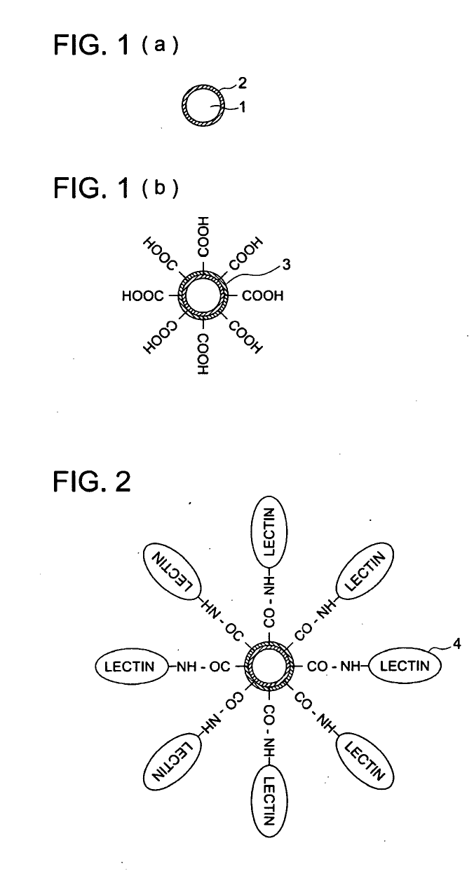

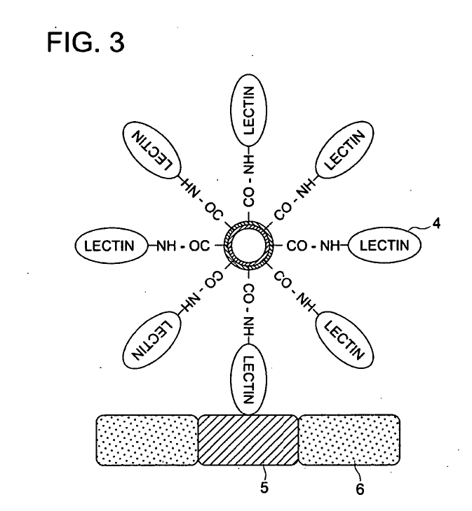

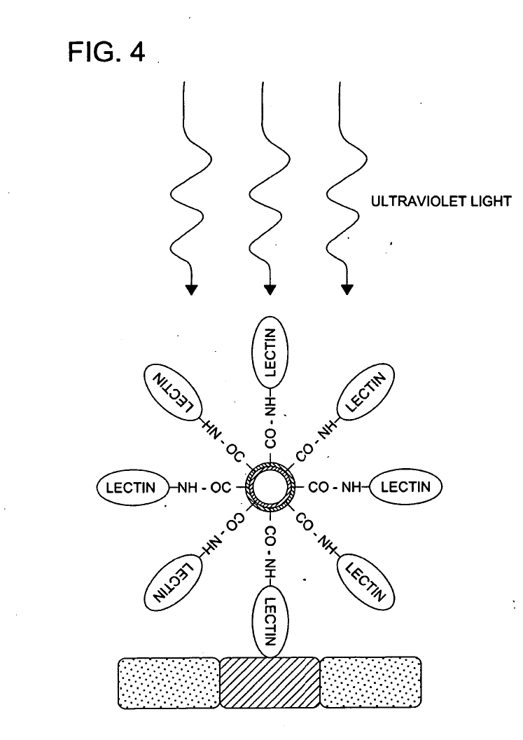

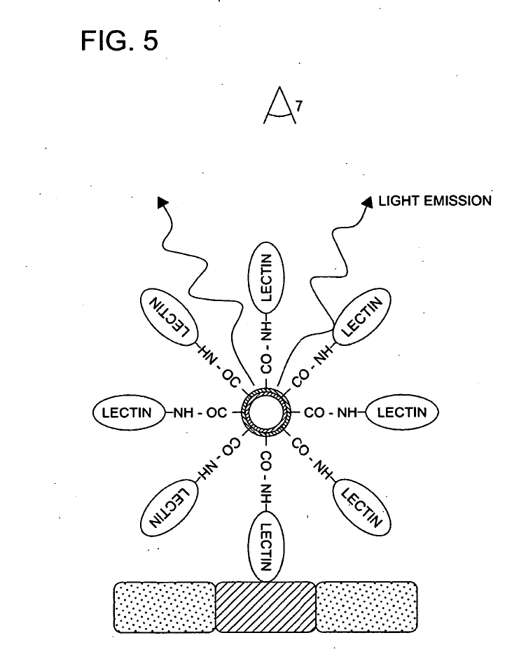

Nanoparticles are applied to the bio-field as a fluorescent labeling substance used for analysis of the behavior of different genes proteins in a cell which have been dyed with plural colors (as described in non-patent document 1). FIGS. 1(a) and 1(b) illustrate examples of a semiconductor nanoparticle used in such a field and having a core/shell structure. On the overall surface of this semiconductor nanoparticle is formed a surface modification layer 3 comprised of a molecule containing a carboxyl group to introduce, to the semiconductor nanoparticles, a lectin as emitting fluorescence suitable for analysis such as detection of cancer and rendering it difficult to inhibit motion of the targeted biosubstance. Herein, the nanorod is generally a cylindrical form of a 2-50 nm length and in the cylindrical form, the diameter of the base end surface is defined as a diameter of the nanorod of the invention. In cases when a nanorod is an oval sphere, the shortest minor diameter is defined as the nanorod diameter of the invention.

Such an average particle size or average rod diameter can be determined through observation by TEM (transmission electron microscope) and the average of measurement values obtained by observation of at least 200 particle images is employed therefor.

Materials constituting the foregoing nanosized particulates are not specifically limited and examples thereof include I-VII group compound semiconductors such as InAs, II-VI group compound semiconductors such as CdS and CdSe, III-V group compound semiconductor such as InAs, IV group semiconductors such as Si and there can also be optimally chosen crystals of these compound semiconductors. Of these, in the invention, the use of a semiconductor nanoparticle formed of Si is suitable without using materials having concerns regarding environmental pollution or toxicity to the human body and in terms of achieving superior emission. With regard to the core/shell constitution, there can be chosen a suitable combination according to employed semiconductor nanoparticle, for example, CdSe-core/ZnS-shell and Si-shell/SiO₂-shell.

In the semiconductor labeling substance of the invention, a modification group to bind specifically to a biosubstance such as a protein, nucleic acid or antigen exists on the surface of the nanosized particulate (the shell surface of a nanoparticle or nanorod). The modification group contains at least a site capable of direct-binding specifically to a biosubstance such as a protein, nucleic acid or antigen (hereinafter, also denoted as biosubstance binding site) and a site directly bound to the surface of the nanosized particulate (hereinafter, also denoted as surface binding site), which may further contain an intermediate site linking the biosubstance binding site and the surface binding site. The fluorescent labeling substance of the invention becomes capable of be bound to a biosubstance as a target of labeling through such a modification group.

The biosubstance binding site may be appropriately adopted depending on the use of a fluorescent labeling in the targeting analysis and its embodiment is not specifically limited. For example, lectin or an antigen used for detection of cancer cells, single strand (ss) DNA for use in detection of DNA in the hybridization method and proteins such as biotin, adipin or antibodies for use in detection of proteins in the ELISA method can be adopted as the biosubstance binding site of the invention.

Meanwhile, examples of a compound forming the surface binding site include a CH₃ group as one of an alkyl group, or SiC, SiOCH, SiCNH and the like, as a compound containing C having an unbonded bond; SiNH, SiCNH and the like as an amino group containing compound; and a silane coupling agent such as (COOH)—Si(OCH₃)₃ as an organic compound containing a carboxyl group. The foregoing carboxyl group or amino group may be introduced by allowing a compound containing such a functional group (e.g., a silane coupling agent) to bind to the nanoparticle surface or in such a manner that a compound not containing such a functional group is allowed to bind to the nanoparticle surface, followed by formation of a carboxyl or amino group through reaction.

Of the foregoing, SiOCH is a compound formed by replacing a part of the matrix of SiO₂ by a methyl group and after such a methyl group is oxidized to form a carboxyl group (carboxylation reaction), a biosubstance binding site may be introduced thereto by a method using an amido-bond, as described later. Similarly, SiC and SiCNH can also introduce a biosubstance binding site through a carboxyl group.

SiNH is a compound containing an amino group formed by replacing a part of amorphous Si₃N₄ by a hydrogen atom. Similarly to the foregoing, a biosubstance binding site can be introduced through a non-binding bond of N or a bond via an amino group. SiCNH can also similarly introduce a biosubstance binding site through an amino group.

Such a compound, in cases when employing the photo-CVD method, exists on the shell surface of the core/shell nanoparticulate in such a form that an island portion in a so-called island/sea structure is layered on the half-face side of a spherical particulate. A compound such as SiC, SiOCH, SiNH or SiCNH and a compound forming a shell are bonded mainly through a covalent bond of Si. Accordingly, it is presumed that a strong bond with SiO₂ is formed through O.

The fluorescent labeling substance may contain modification groups other than the above-described ones, for example, a modification group to enhance hydrophilicity, within a range of not inhibiting the effect of the invention.

### Region Having No Modification Group

The area of a region having no modification group on the particulate surface of the fluorescent labeling substance of the invention preferably accounts for at least 50% of the total surface area of the nanosized particulates to attain sufficient fluorescence visibility for analysis and also not to adversely affecting bonding to the targeted biosubstance. For instance, when a fluorescent labeling substance bound to an affected area (such as a cancer cell) is observed from above, if at least the upper half of a spherical nanoparticle has a light-emitting function, there is no need to have a modification group.

In the invention, “an area of the region having no modification group” refers to the area of a region which is not covered with a molecule forming the modification group and which can be measured through observation by using a TEM.

Such an area having no modification group is preferably formed continuously on the nanoparticulate surface. For example, a reaction of introducing a modification group is allowed to proceed only on half-surface of a nanoparticle by the method as described later, thereby enabling to secure a continuous region having no modification group on the opposite half-spherical side. Similarly, a modification group may be allowed to exist only on one half of the cylindrical surface of a nanorod or on one side of top and bottom parallel surfaces of the nanorod. “Cylindrical surface” and “one side of parallel surfaces” of a nanorod refer to the side surface and one of parallel surfaces of a cylindrical nanorod. A nanorod of an oval sphere is presumed to be a pseudo-spherical form and its half spherical surface is ascribed to be a half-surface of a cylindrical surface, and such a nanorod is presumed to have no parallel surface.

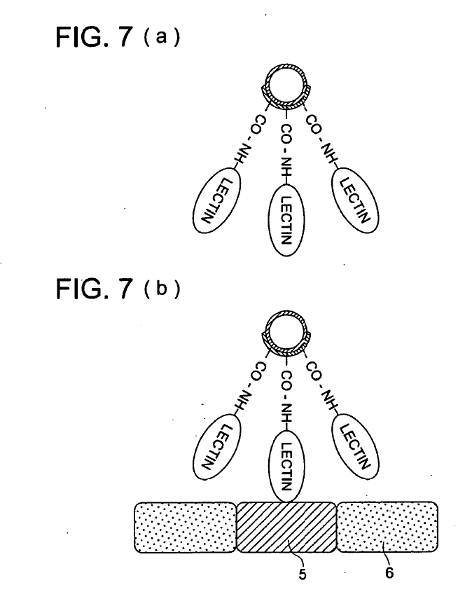

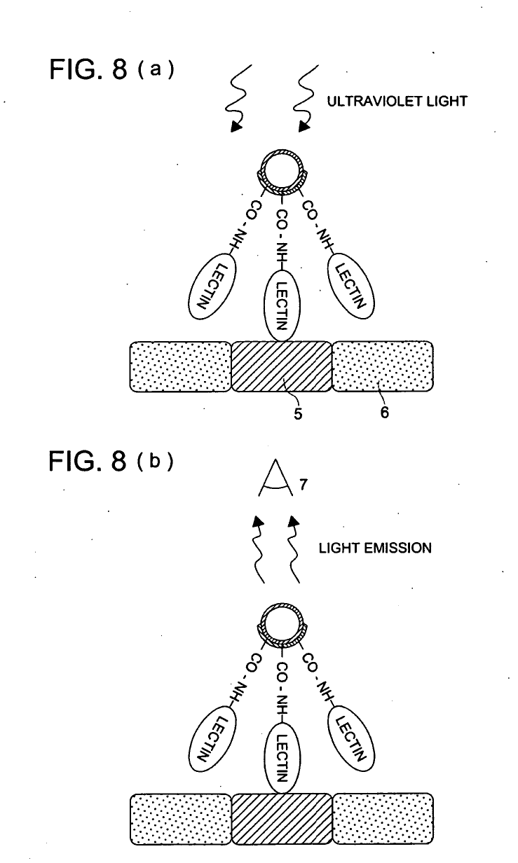

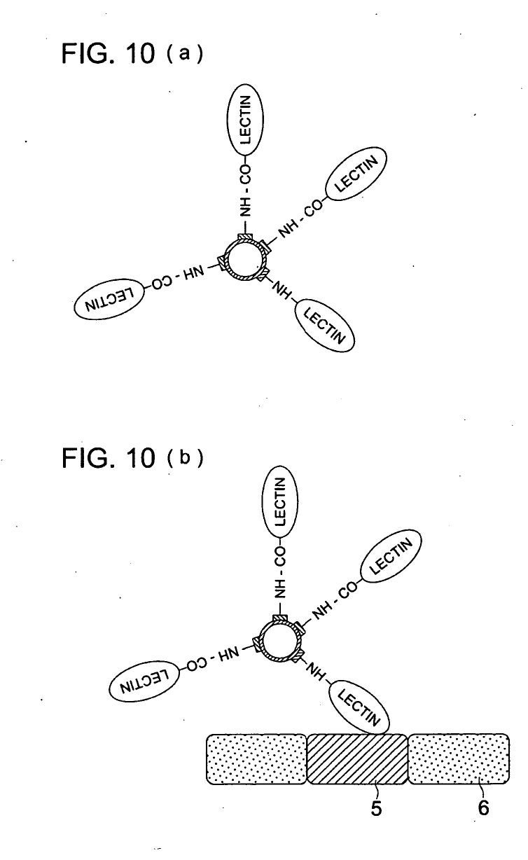

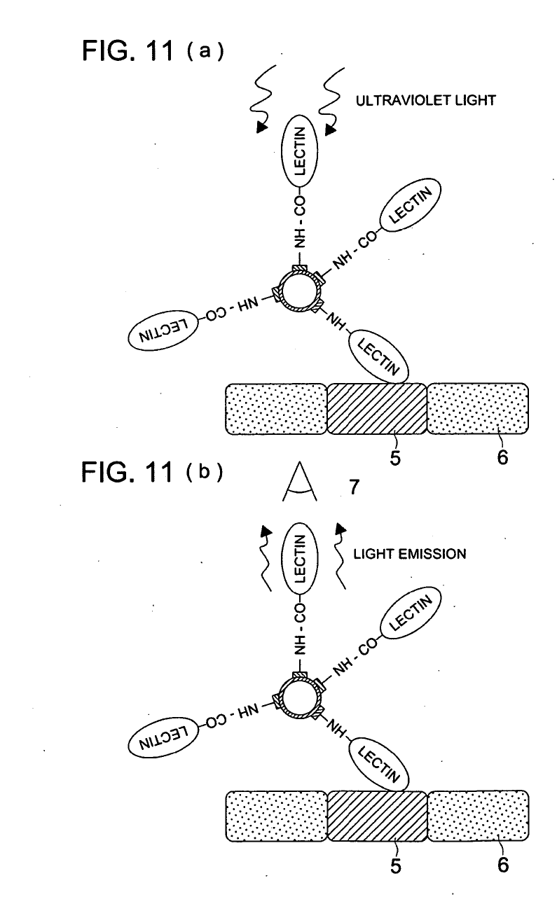

When such a nanoparticulate, as described above is bonded to a biosubstance [FIGS. 7(b) and 10(b)], an exciting light incoming to the nanoparticulate is difficult to be shielded by a modification group [FIGS. 8(a) and 11(a)] and fluorescent light emitted from the nanoparticulate reaches a fluorescence detector without being shielded by the modification group [FIGS. 8(b) and 11(b)]. Accordingly, a substantial light-emitting efficiency, which is defined as a ratio of the number of photons detected in a fluorescence detector to that of photons ejected from an exciting light irradiation apparatus, is enhanced, compared to the conventional nanoparticulates in which a modification group is introduced on the overall surface, thereby achieving superior detection accuracy. Therefore, the above-described nanoparticulates of the invention is suitably applicable to analysis employing conventional fluorescent labeling substances.

### Preparation Method of Fluorescent Labeling Substance

### Preparation of Nanoparticulate:

Inorganic fluorescent nanoparticles usable in the invention can be prepared in accordance with commonly known methods. The preparation method is not specifically limited but examples thereof include gas phase processes such as a CVD method, a laser ablation method, a silane degradation method and a Si electrode vaporization method, and liquid phase processes such as an electrolysis method and a reversed micelle method. Inorganic nanoparticles prepared by these methods may be suspended in liquid or fixed on a plate, but any form is applicable so long as introduction of a modification group is feasible.

### Introduction of Modification Group:

The modification group of the invention can be introduced to the nanoparticulate surface, for example, in such a manner that a compound forming a surface binding site is introduced onto the nanoparticulate surface and a material forming a biosubstance binding site is allowed to bind to this compound. Alternatively, a compound forming a spacer is allowed to bind to a compound forming a surface binding site and further thereto, a material forming a biosubstance binding site may also be allowed to bind.

Introduction of a surface binding site is not limited to a specific method but can be achieved by appropriate methods but the use of a photo-CVD method, as described below, is cited as the preferred embodiment of the invention in terms of a modification group being easily introduced to a selected region.

### Embodiment 1

A core/shell nanoparticle comprised of a Si core and a SiO₂ shell is prepared and the nanoparticle is fixed on the planar surface. Subsequently, light is irradiated from only one direction and one side of the nanoparticle is exposed thereto in an atmosphere of SiH(CH₃)₃ and N₂O and is allowed to react by a photo-CVD method. Thereby, a layer composed of SiOCH, which corresponds to a layer obtained by replacing a part of a SiO₂ matrix with an alkyl group, for example, CH₃ (methyl group), is formed to cover half of the spherical SiO₂ shell surface. Further, oxidation of CH₃ under an atmosphere of CO₂ converts the methyl group to a carboxyl group.

### Embodiment 2

Using a core/shell nanoparticle of a Si core and a SiO₂ shell, light is irradiated from only one direction and the nanoparticle is exposed thereto in an atmosphere of SiH₄ and NH₃ and is allowed to react by a photo-CVD method. Thereby, a layer comprised of amorphous Si₃N₄ containing many hydrogen atoms, that is, SiNH having NH₂ (amino group) is formed to cover a part of the spherical SiO₂ shell surface.

### Embodiment 3

Using a core/shell nanoparticle comprised of a Si core and a SiO₂ shell, light is irradiated from one direction and the nanoparticle is exposed thereto in an atmosphere of C₄F₈—C₂H₂ and is allowed to react by a photo-CVD method. Thereby, a layer having an amorphous C—H membrane, that is, CH₃ (methyl group) is formed so as to cover a part of the spherical SiO₂ shell surface. Similarly to the foregoing embodiment 1, oxidation of CH₃ in an atmosphere of CO₂ converted the methyl group to a carboxyl group.

### Embodiment 4

There is prepared a core/shell nanorod comprised of a Si core and a SiO₂ shell, in which a Si nanorod is vertically formed on a Si substrate through microfabrication and oxidized with O₂ to form Si/SiO₂. Then, etching the Si substrate side, the nanorod is separated from the substrate and heated in an atmosphere of NH₃ to convert only the Si portion of the bottom surface to SiNH. Thereby, a layer comprised of SiNH and containing NH₂ (amino group) is formed only on the bottom surface.

In the invention, an organic molecule, called a bi-functional cross-linker, such as SMCC (sulfomaleimidomethylcyclohexanecarboxylic acid sulfohydroxysuccinimide ester sodium salt) may be linked as a spacer.

The foregoing SMCC has two functional sites exhibiting directivity to an amino or thiol group, and one of them is allowed to link, for example, SiNH and the other one can be used for bonding to a compound to form a biosubstance binding site. Further, there can also be usable a bifunctional cross-linker having a structure in which a material to form a surface binding site and a material to form a biosubstance bonding side are introduced to both ends of an oxyalkylene, such as polyethylene glycol (PEG).

A biosubstance binding site, in which the above-described compound to form the surface binding site or a functional group capable of bonding to a functional group contained in a bifunctional cross-linker is preliminarily introduced to a part of ssDNA, adipin, biotin or an antibody by commonly known means, can be introduced to a modification group. For example, when a nanoparticle having introduced a carboxy group and lectin having introduced a carboxyl group are allowed to react, biotin is introduced to a modification group through a peptide bonding. Similarly, when a nanoparticle having introduced an amino group and lectin having introduced a carboxyl group are reacted, biotin is introduced to a modification group through peptide bonding.

### EXAMPLES

The present invention will be further described with reference to examples, but the invention is by no means limited to these.

### Example 1

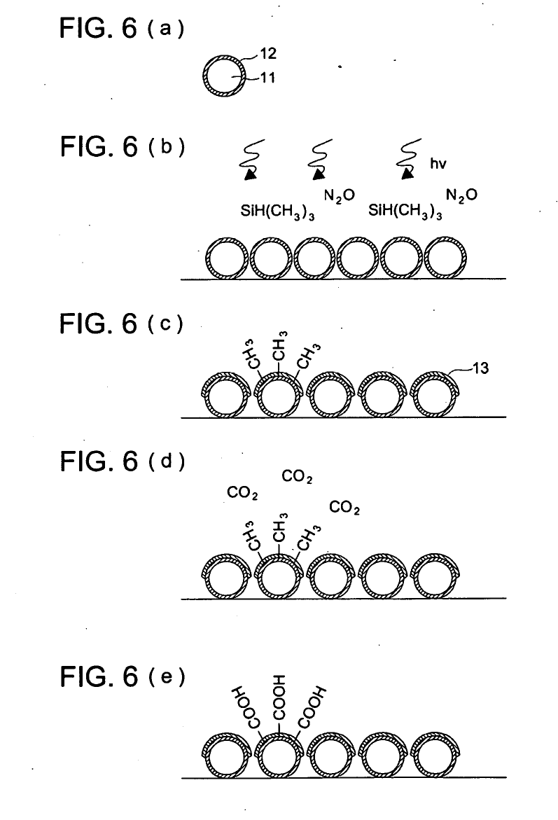

First, in accordance with the known method (JP-A No. 5-224261), core/shell nanoparticles comprised of a 2 nm diameter Si core and a 1.5 nm thick SiO₂ shell were prepared by a microwave plasma decomposition method of SiH₄ gas and an oxidation treatment by a strong-alkali treatment, as shown in FIG. 6(a). The obtained nanoparticles were exposed to an ultrasonic treatment in water to become separated from the substrate and the nanoparticles, which exhibited strong hydrophobicity, formed a monoparticulate layer on the water surface, which was dried, thereby, the nanoparticles were disposed on the substrate. Instead of the method of this example, nanoparticles may be conveyed by a gas carrier and electrostatically adsorbed onto an electrostatic chuck. Subsequently, in a CVD apparatus, an excimer laser was irradiated from one side under an atmosphere at a flow rate ratio of SiH(CH₃)₃ and N₂O of 1:1, a pressure of 666 Pa and a temperature of 350° C. and one half side of the nanoparticles were exposed thereto, as shown in FIG. 6(b) and reacted through the photo-CVD method. Thereby, a layer composed of SiOCH in which a part of the SiO₂ matrix was replaced by CH₃ (methyl group) was formed so as to cover one half of the spherical surface of the SiO₂ shell, as shown in FIG. 6(c). Further, exposure to an atmosphere of CO₂, as shown FIG. 6(d) and heating at a high temperature of about 900° C. converted the foregoing methyl group to a carboxyl group, as shown FIG. 6(e). Subsequently, this carboxyl group was bonded to an amino group of LECTIN through a peptide linkage to obtain a fluorescent labeling substance in which the LECTIN was bonded to half of the spherical surface, as shown in FIG. 7(a). The thus obtained fluorescent labeling substance of the invention, having formed modification groups only on the half of the spherical surface, exhibited a fluorescence having a peak near 600 nm when exposed to ultraviolet light of 250 nm and its efficiency increased to approximately 1.4 times the fluorescent labeling substance having formed modification groups on the entire spherical surface.

### Example 2

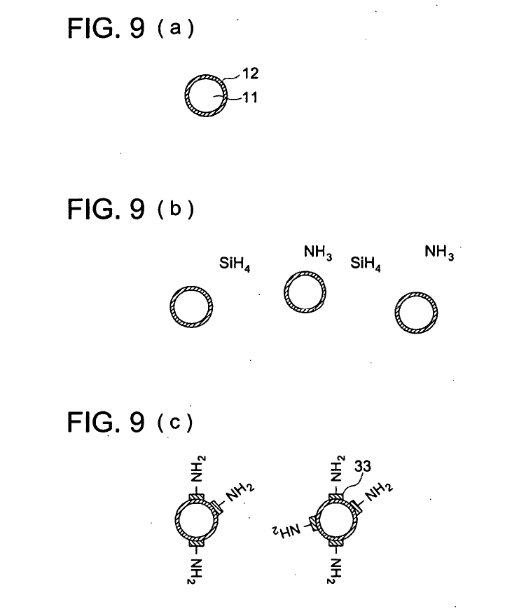

First, in accordance with the known method (JP-A No. 5-224261), core/shell nanoparticles comprised of a 2 nm diameter Si core and a 1.5 nm thick SiO₂ shell were prepared by an oxidation treatment of a microwave plasma decomposition method of SiH₄ gas and a strong-alkali treatment, as shown in FIG. 9(a). The obtained nanoparticles were immersed in a surfactant solution of Tween 80 to make their surfaces hydrophilic and dispersed in the form of micro-droplets by an ultrasonic treatment. Then, inert gas He was introduced thereto to perform vaporization in a vaporizer. Thereby, Si/SiO₂ nanoparticles, enveloped by Tween 80 were introduced by the He gas as a carrier into a CVD apparatus. As shown in FIG. 9(b), a plasma CVD treatment was performed over about 3 sec. in a CVD apparatus with applying an electric power of 400 W at a flow rate of SiH₄ and NH₃ of 1:3 and a temperature of 400° C. Thereby, a SiNH membrane which contained a unbonded bond of N or amino group such as NH₂ or NH, formed by the plasma treatment, were formed to cover at most half of the nanoparticle surface, as shown in FIG. 9(c).

Subsequently, this amino group or unbonded bond of N was linked to a carboxy group of LECTIN through peptide linkage to obtain a fluorescent labeling substance in which the LECTIN was bonded to a part of the spherical surface, as shown in FIG. 10(a). The thus obtained fluorescent labeling substance of the invention, having formed modification groups only on not more than half of the spherical surface, exhibited a fluorescence having a peak near 600 nm when exposed to ultraviolet light of 250 nm and its efficiency increased to approximately 1.2 times that of the fluorescent labeling substance having formed modification groups on the entire spherical surface.

### Example 3

Similarly to the treatment conditions of the photo-CVD of Example 1 as shown in FIG. 6(b), reaction was performed through photo-CVD, provided that instead of SiH(CH₃)₃ and N₂O, C₄F₈ and C₂H₂ were used at a flow rate ratio of C₄F₈ and C₂H₂ of 1:3, and an excimer laser was irradiated from only one side to the half side of nanoparticles under an atmosphere of a pressure of 400 Pa and a temperature of 400° C. Thereby, an amorphous carbon membrane containing CH₃ (methyl group), unbonded CH and the like was formed instead of the SiOCH membrane of FIG. 6(c). Further heating at about 900° C. under an atmosphere of CO₂ converted the foregoing methyl group or non-bonding CH group to a carboxyl group. Subsequently, this carboxyl group was bonded to an amino group of LECTIN through a peptide linkage to obtain a fluorescent labeling substance in which the LECTIN was bonded to only half of the spherical surface. The thus obtained fluorescent labeling substance of the invention, having formed modification groups only on the half of the spherical surface, exhibited a fluorescence having a peak near 600 nm when exposed to ultraviolet light of 250 nm and its efficiency increased to approximately 1.4 times that of the fluorescent labeling substance having formed modification groups on the whole spherical surface.

### Example 4

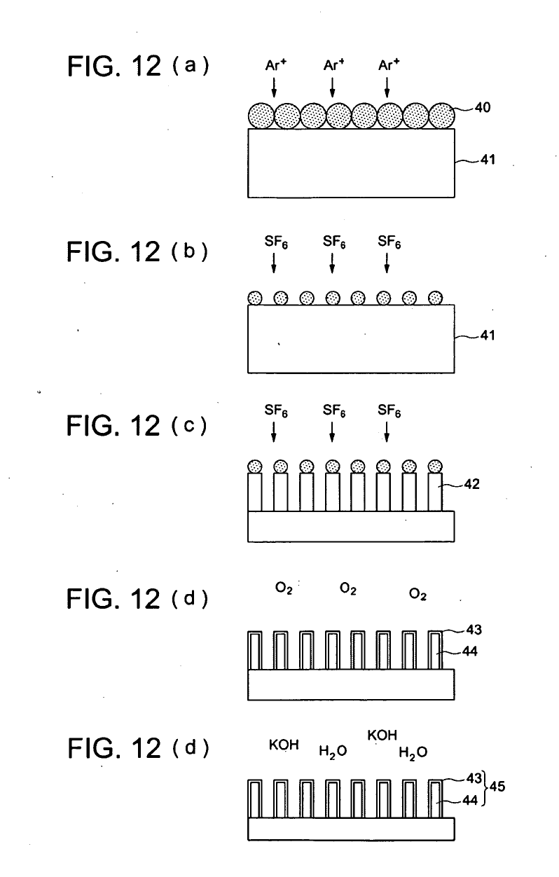

Si nanorods were prepared in accordance with the known method [as disclosed in J. Rose et al., Mat. Res. Symp. Proc. Vol. 832 (2005), F7.14.1]. First, a solution, in which surfactant Triton X-100 was dissolved in a mixture of water and methanol at a ratio of 1:400 and polystyrene of a 300 nm diameter sphere was further dissolved therein, was coated on a Si (111) substrate and allowed to stand in a desiccator for one day. Thereby, the structure of the Si substrate was covered with a polystyrene sphere monolayer. Subsequently, etching by Ar⁺ was performed with applying a pressure of 10.7 Pa to make the size of polystyrene spheres smaller to form a mask used for the subsequent step. As shown in FIG. 12(b), reactive ion-etching was performed by applying an electric power of 50 W and using SF₆ and He at a gas ratio of 1:3 [FIG. 12(c)]. After conducting cleaning with methanol, as shown in FIG. 12(d), oxidation was performed in O₂ at about 900° C. and 1000 sccm to form core/shell nanorods of Si/SiO₂. Then applying an ultrasonic treatment in an aqueous KOH solution, the Si portion at the base of the nanorods was etched to separate the Si/SiO₂ nanorods from the substrate, as shown in FIG. 12(e). Thereby, nanorods with a diameter of 50 nm and a length of 200 nm were formed.

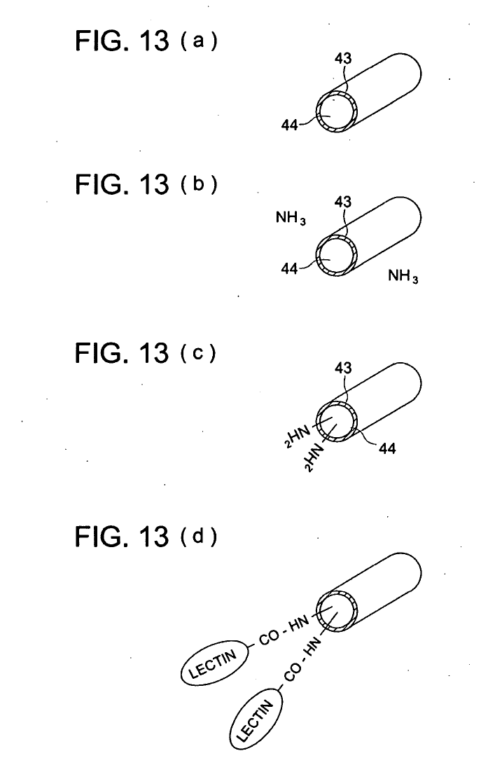

In the structure shown in FIG. 13(a), Si became exposed at its base portion. As shown in FIG. 13(b), heating at 700° C. in an atmosphere of NH₃ at 1 atmosphere nitrided only this exposed Si portion to form SiNH (46) and to form a NH₂ bond on only one parallel surface, as shown in FIG. 13(c). In the thus formed nanorod, this amino group was allowed to bind to a carboxyl group of LECTIN through a peptide linkage to obtain a fluorescent labeling substance in which LECTIN was bonded to only a part of the spherical surface of the nanorod. The thus obtained fluorescent labeling substance of the invention, having formed modification groups only on one parallel surface of the rod, exhibited a fluorescence having a peak near 600 nm when exposed to ultraviolet light of 250 nm and its efficiency increased to approximately 2 times that of a fluorescent labeling substance having formed modification groups on the entire spherical surface.

### Example 5

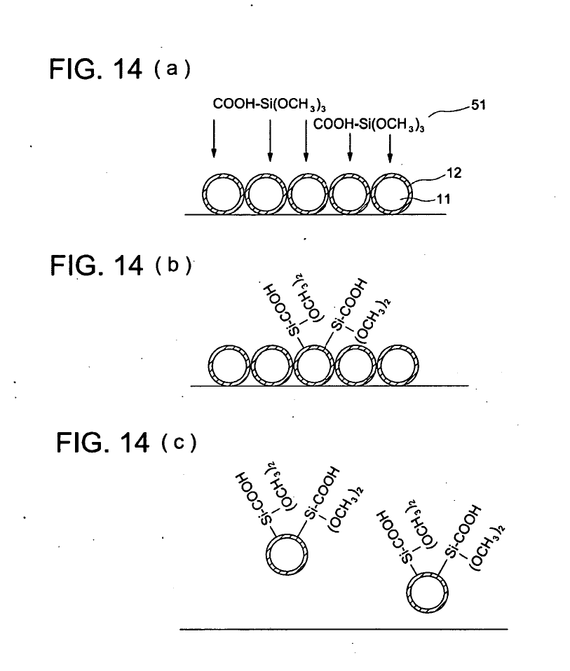

In accordance with the known method (as described in JP-A No. 5-224261), core/shell nanoparticles having a 2 nm diameter core and a 1.5 nm thick shell were prepared through a microwave plasma decomposition method of SiH₄ gas and oxidation by a strong alkali treatment, as shown in FIG. 14(a). The obtained nanoparticles were exposed to an ultrasonic treatment in water to become separated from the substrate and the nanoparticles, which exhibited strong hydrophobicity, formed a monoparticulate layer on the water surface, which was dried, and thereby, the nanoparticles on the substrate were disposed. Subsequently, a solution of COOH—Si(OCH₃)₃ as a silane coupling agent was sprayed thereto, as shown in FIG. 14(a) and heated at 120° C., whereby only half of the surface of the core/shell nanoparticles was cross-linked by Si to allow COOH—Si(OCH₃)₃ to be bonded, as shown in FIG. 14(b). Finally, this carboxyl group was allowed to bind to an amino group of LECTIN through a peptide linkage to obtain a fluorescent labeling substance in which the LECTIN was bonded to only half of the spherical surface, as shown in FIG. 14(c). The thus obtained fluorescent labeling substance of the invention, having formed modification groups only on half of the spherical surface, exhibited a fluorescence having a peak near 600 nm when exposed to 250 nm ultraviolet light and its efficiency increased to approximately 1.4 times that of a fluorescent labeling substance having formed modification groups on the entire spherical surface.

1-12. (canceled)

13. A fluorescent labeling substance comprising nanoparticles or nanorods having a modification group capable of binding to a biosubstance on surfaces of the nanoparticles or nanorods and a region not having the modification group is provided on the surfaces.

14. The fluorescent labeling substance as claimed in claim 13, wherein the region not having the modification group accounts for at least 50% of total surface area.

15. The fluorescent labeling substance as claimed in claim 13, wherein the modification group capable of binding to a biosubstance is present on a half of a spherical surface of the nanoparticles.

16. The fluorescent labeling substance as claimed in claim 13, wherein the modification group capable of binding to a biosubstance is present on a half of a cylindrical surface of the nanorods.

17. The fluorescent labeling substance as claimed in claim 13, wherein the modification group capable of binding to a biosubstance is present on one of parallel surfaces of the nanorods.

18. The fluorescent labeling substance as claimed in claim 13, wherein the modification group capable of binding to a biosubstance is introduced through reaction with a COOH (carboxyl) group introduced by carboxylation of an alkyl group or a substance containing C having an unbonded bond, formed on the surfaces of the nanoparticles or the nanorods.

19. The fluorescent labeling substance as claimed in claim 18, wherein the alkyl group or the substance containing C having an unbonded bond, formed on the surfaces of the nanoparticles or the nanorods is SiC, SiOCH or SiCNH.

20. The fluorescent labeling substance as claimed in claim 13, wherein the modification group capable of binding to a biosubstance is introduced by forming a substance containing a COOH (carboxyl) group or an amino (NH₂) group on the surfaces of the nanoparticles or the nanorods, followed by reaction with the COOH group or the NH₂ group.

21. The fluorescent labeling substance as claimed in claim 20, wherein the material containing a NH₂ (amino) group is SiNH or SiCNH.

22. The fluorescent labeling substance as claimed in claim 13, wherein the modification group capable of binding to a biosubstance is a modification group capable of binding specifically to a protein, a nucleic acid or an antigen.

23. The fluorescent labeling substance as claimed in claim 13, wherein the nanoparticles or the nanorods comprise a core comprising a semiconductor nanocrystal and a shell comprising a substance exhibiting a band gap greater than that of the core.

24. The fluorescent labeling substance as claimed in claim 13, wherein the nanoparticles exhibit an average particle diameter of 2 to 50 nm or the nanorods exhibit an average diameter of 2 to 50 nm.
